(19) United States
(12) Patent Application Publication
(10) Pub. No.: US 2008/0078389 A1
(43) Pub. Date: Apr. 3, 2008

(54) HELIOX DELIVERY SYSTEM AND METHOD WITH POSITIVE PRESSURE SUPPORT

(76) Inventors: Yang Xiao, Williamsville, NY (US); Stephan Gamard, Kenmore, NY (US)

Correspondence Address: PRAXAIR, INC.;LAW DEPARTMENT - M1 557, 39 OLD RIDGEBURY ROAD, DANBURY, CT, 06810-5113, US

(21) Appl. No.: 11/861,727

(22) Filed: Sep. 26, 2007

Related U.S. Application Data
(60) Provisional application No. 60/848,615, filed on Sep. 29, 2006.

(51) Int. Cl. A61M 16/12

(52) U.S. Cl. 128/204.22

(57) ABSTRACT

A Heliox delivery system with positive pressure support is disclosed. The positive pressure support Heliox delivery system comprises at least two inlet ports, one of which is coupled to a source of Heliox gas and another coupled to a source of oxygen gas. The system further includes a gas blender, one or more control valves, a gas analyzer, an outlet port, a breathing circuit and a control unit. The control unit is adapted to operatively control both the gas blender to adjust the blending of oxygen and Heliox and the control valve to adjust the flow rate or pressure of the blended gas mixture in response to user inputs as well as the measured parameters from the gas analyzer, flow meters and associated sensors.

### CROSS REFERENCE TO RELATED APPLICATIONS

This application claims priority to U.S. provisional patent application Ser. No. 60/848,615 filed Sep. 29, 2006 the disclosure of which is incorporated by reference herein.

### FIELD OF THE INVENTION

The present invention relates to a medical gas delivery device and more particularly, to a system and method for mixing helium gas or Heliox gas with oxygen gas and delivering the gas mixture into the pulmonary system of a patient.

### BACKGROUND OF THE INVENTION

Heliox is a gas mixture of helium and oxygen and is commonly used in hospital respiratory applications, both in the emergency and intensive care units. There are, however, few dedicated devices or means to deliver Heliox gas to the patient. For instance, the various instruments used to deliver Heliox gas to a patient including off-the shelf blenders, flow meters, nebulizers, etc. have typically been designed and optimized for use with a heavier medical gas like oxygen or air. A particular disadvantage of the prior art instruments and devices used to administer Heliox to patients is a lack of information concerning the Heliox gas composition being administered by such devices. Since nitrogen and helium tanks use the same CGA connections, a mixture of nitrogen and oxygen might be, and on occasion have been inadvertently given to the patient instead of Heliox. In addition, most flow meters and other electronic instruments used in hospital environments are typically only calibrated for use with oxygen or air or to a given specific Heliox mix such as 80% helium/20% oxygen (heliox 80/20). Thus, the respiratory therapist or nurse must often use a conversion chart to correlate the flow meter reading to the actual flow of gas when using Heliox. This reliance on conversion charts and associated correlation practice is neither precise nor convenient since the practice is susceptible to human error and inaccurate conversion charts.

Other dedicated Heliox delivery systems are large, costly and complex devices. For example, U.S. Pat. No. 5,429,123 (Shaffer) discloses a method and system for controllably introducing gaseous mixtures, including a blend of helium and oxygen, into the pulmonary system of patients with a feedback control system based on the patient's oxygen saturation level. However, the Shaffer reference discloses a design for the ventilator which mechanically controls the patient's inspiratory and expiratory breathing cycle, normally through an invasive medical procedure as intubation.

What is needed is a reliable, an easy to use, non-invasive Heliox delivery system and method.

### SUMMARY OF THE INVENTION

The present invention may be characterized as a positive pressure support Heliox delivery system. The present Heliox delivery system comprises: at least two inlet ports, one of which is coupled to a source of helium containing gas and another coupled to a source of oxygen containing gas; a gas blender in flow communication with the two inlet ports; a flow valve disposed downstream of the gas blender; a gas analyzer, flow meter and a breathing circuit including at least one pressure sensor disposed downstream of the flow valve; and a control unit adapted to receive inputs from the gas analyzer, the pressure sensor, the flow meter, and user inputs specifying desired gas concentrations and desired gas delivery pressure conditions, the control unit further adapted to operatively control the gas blender to adjust the blending of oxygen gas and helium containing gas and to control the flow valve to adjust the blended gas flow rate and pressure in response to the inputs from the gas analyzer, pressure sensor, flow meter and user inputs.

The present invention may also be characterized as a method for delivering Heliox to a patient via a breathing circuit, the method comprising the steps of: (i) blending a helium containing gas and oxygen gas; (ii) controlling the flow rate and pressure of the blended gas; (iii) analyzing the concentrations of oxygen and helium in the blended gas; (iv) ascertaining the pressure and flow rate in the breathing circuit; (v) operatively controlling the blending of the helium containing gas and oxygen gas in response to the ascertained pressure and flow rate in the breathing circuit; and (vi) further adjusting the flow rate and pressure of the blended gas mixture delivered to a patient in response to the ascertained pressure and flow rate in the breathing circuit.

### BRIEF DESCRIPTION OF THE DRAWINGS

While the specification concludes with one or more claims distinctly pointing out the subject matter that applicants regard as their invention, it is believed that the invention will be better understood when taken in connection with accompanying drawings in which:

FIG. 1 is a schematic diagram of an embodiment of the Heliox delivery system with positive pressure support;

FIG. 2 is a schematic diagram of an alternate embodiment of the Heliox delivery system adapted to operate in a BiLevel Positive Airway Pressure (BiPAP) configuration;

FIG. 3 is a schematic diagram of an embodiment of the positive pressure support Heliox delivery system used in conjunction with a nebulizer for delivery of drugs to a patient; and

FIG. 4 is a schematic diagram of another embodiment of the positive pressure support Heliox delivery system used in conjunction with a nebulizer for delivery of drugs to a patient.

### DETAILED DESCRIPTION

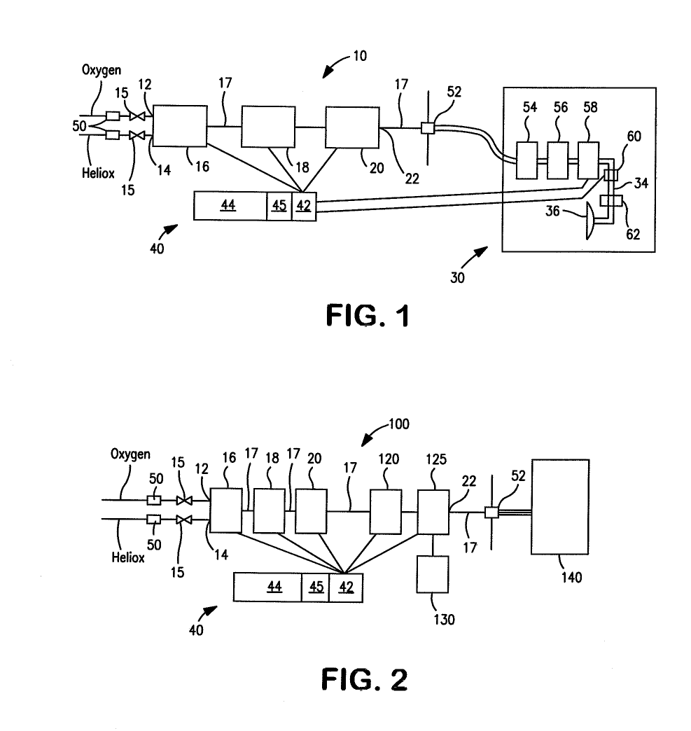

With reference to FIG. 1, there is shown a schematic diagram of an embodiment of a positive pressure support Heliox delivery system (10) that can blend Heliox gas and oxygen gas to attain a prescribed percent concentration of oxygen in the gas delivered to a patient. The illustrated positive pressure support Heliox delivery system (10) comprises at least two inlet ports (12, 14), one of which is coupled to a source of helium or Heliox gas and another coupled to a source of oxygen or oxygen containing gas such as air. The Heliox delivery system (10) also includes a gas blender (16), one or more control valves (18), a gas analyzer (20), an outlet port (22), a breathing circuit (30) and a control unit (40). The preferred control unit (40) includes a microprocessor based controller (42), a display (44), and user interface (45). The Heliox delivery system (10) may also include one or more alarms (50), a filter (52), a heater (54), a humidifier (56), and a flow meter (58).

In the illustrated embodiment, oxygen gas and helium containing gas are supplied to the positive pressure support Heliox delivery system (10) via inlet ports (12, 14). The helium containing gas can be supplied via a commonly available gas mixture of helium and oxygen or can be pure helium. The ratio of helium and oxygen concentration within commercially available Heliox mixtures is typically about 80% helium and about 20% oxygen (80/20) or about 70% helium and about 30% oxygen (70/30), although other concentrations may also be used. The Heliox gas and oxygen gas originate from facility gas sources (not shown) and are delivered to the usage site via an internal gas circuit (not shown). Alternative gas sources for the Heliox and oxygen gases many include gas cylinders, gas tanks, or other gas delivery route. The shown gas circuit typically includes one or more gas pressure regulators (15) to deliver the Heliox and oxygen gases via the inlet ports (12, 14) at a pressure of about 50 psi.

An alarm (50) is operatively coupled to the Heliox gas source upstream of the regulator and to the control unit to inform the user when the pressure in the Heliox line falls below a certain level, 500 psi for instance. The alarm (50) can be visual indicator such as a Light Emitting Diode (LED) and/or an audible indicator such as a chime, tone or whistle. A shutoff means can be operatively linked to the alarm to automatically stop the Heliox gas flow or bypass the gas blender in order to deliver 100% pure oxygen to the patient in the event the alarm (50) is triggered. Where Heliox gas cylinders are used, an alarm indicator directly connected to the source of Heliox would let the user or respiratory therapist know when to change Heliox cylinders. Likewise, an alarm (50) may also be operatively coupled to the oxygen source upstream of the regulator and also electronically coupled to the control unit to inform the user when the pressure in the oxygen line falls below a certain level. Alternatively, the user can set up the alarm to be activated based on adverse gas concentration levels, oxygen saturation levels in the patient, system flow rates, pressures, temperatures, and humidity levels.

The positive pressure support Heliox delivery system (10) includes a gas blender (16) that effectively blends the oxygen and Heliox gases at or near the desired point of use. The gas blender (16) is operatively coupled to the control unit (40) which precisely controls the final oxygen level (FiO₂) or the concentration of oxygen in the blended gas (17) entering the lungs of the patient from a minimum of about 10% to the upper limit of about 100%. In the preferred embodiment, an actuator is used for controllably driving flow control valves within the gas blender (16) to various selected blending positions so as to adjust and meter the incoming flows of Heliox and oxygen gases to achieve the desired final oxygen level (FiO₂) in the blended gas (17). An example of such internal flow control valves may take the form of a disk-like orifice plates having a plurality of peripheral orifices that are calibrated for particular flow rates of the Heliox and/or oxygen gases. The actuator will move each of the disk-like orifice plates to the prescribed positions based on user selected inputs of the desired final oxygen level (FiO₂).

The blended gas (17) exits the gas blender (16) and then passes through one or more control valves (18) which operatively control the final pressure and flow of the blended gas (17). Preferably, the blended gas (17) will maintain a positive pressure between about 0 to 30 cm H₂O and a volumetric flow rate of about 0 to 25 liters per minute and a helium concentration from 0 to 90%. The control unit can be programmed to adjust the gas composition, pressure, and flow rate automatically over time or patient's breathing pattern.

The blended gas (17) is then routed to a gas analyzer (20) to determine an accurate measurement of helium and oxygen concentrations within the blended gas flow. Preferably, the gas analyzer (20) uses the thermal properties of helium to analyze the amount of helium in the gaseous mix using thermal conductivity cells. The gas analyzer also independently measures the amount of oxygen with a galvanic oxygen cell, so that the exact concentration of oxygen and helium in the gas mix is known independently of one another. If the gas analyzer indicates there is an anomaly with the gaseous mix (e.g. the relative concentrations do not total or approximate 100%), an alarm should warn the user of the issue and, where appropriate automatically shutoff the system. Measuring the concentration of helium gas and oxygen gas independently should aid in identifying any incorrect tank connection errors, where another gas is substituted for Heliox by mistake. In addition, the independent determination of helium and oxygen can be coupled to the display and alarm systems such that the user can be advised or warned when any concentration of either oxygen or helium drops below a prescribed concentration level.

The blended gas mixture of Heliox and oxygen is subsequently delivered through an outlet port (22) for ultimate delivery to the patient. An optional replaceable filter (52) is also preferably disposed in the flow path proximate the outlet port (22).

In the illustrated embodiment, outlet port (22) is connected to a breathing circuit (30). Breathing circuit (30) is preferably composed of various tubes, adaptors and connectors, such as corrugated tubes, oxygen tubes, CPAP tubes BPAP tubes, or other conduit arrangement to carry the flow of the blended gas to a patient interface (34). The patient interface (34) can include a non-invasive nasal mask, oral mask, cannula, face mask with one way valve to allow expiration, nasal prong, or other mask type device (36) that delivers the blended gas flow to patient's airway. Preferably, a nasal mask type device (36) is used that is capable of operating at positive pressures of up to about 50 cm H₂O. The patient inhales the blended gas through the nasal mask (36) and exhales through the mouth.

Control unit (40) operatively controls both the gas blender (16) to adjust the blending of oxygen and Heliox and the one or more control valves (18) to adjust the flow rate or pressure of the blended gas mixture (17) in response to user inputs as well as measured parameters from the gas analyzer, flow meters and associated sensors. The control unit (40) includes a microprocessor based controller (42), a display (44), and a user interface (45). The microprocessor based controller (42) includes the logic and control algorithms to effect precise control of the gas blender (16) and control valves (18) based on user inputs and other collected data and information. The display (44) is preferably an LCD type screen that displays gas delivery parameters such as flow rate of mixed gas, helium concentration of the blended gas, oxygen concentration of the blended gas, breathing curve of the patient, oxygen saturation of the patient, inspiratory positive pressure support, alarm settings, audible and visual alarms indicators, and auxiliary sensor measurements such as temperature, humidity, pressure, etc. The microprocessor based controller (42) also includes a data exporting port such as USB port, so the patient data can be downloaded.

The user interface (45) is preferably a plurality of dials, keypads, buttons or even icons that would be located on the display (44). User inputs would typically include one or more of the following settings: desired oxygen and helium concentrations, alarm level settings, inspiration positive pressure support, and related parameters.

Also shown in the illustrated embodiment are various auxiliary devices. Such auxiliary devices may include one or more of the following devices: alarm (52), heater (54), humidifier (56), flow meter (58), auxiliary sensors (60), and nebulizer (62), or any combinations or arrangements thereof. Preferred uses of such auxiliary devices are described in more detail below.

The heater (54) is preferably included in the system to warm the blended gas (17). Due to the high thermal conductivity of helium, it is often not advised for patients to inhale a cold gas, due to the risks of hypothermia that can arise. In the preferred embodiment, the heater (54) can be a simple heat wrap around the gas tubing or a heated filament in the tubing.

The humidifier (56) is also preferably included within the illustrated embodiment to deliver a saturated gas mixture of Heliox and oxygen to the patient. The humidification of the blended gas (17) can occur with a jet nebulizer, a bubble humidifier, or a pass-over humidifier, with or without the addition of heat from the heater (54).

The blended gas flow rate is preferably controlled by a flow meter (58). The flow meter (58) is operatively coupled to the control unit (40) and is controlled in response to the analyzed concentrations of helium and oxygen and a measured pressure differential in the flow path. The gas analyzer (16) provides the gas concentration of helium (C_{He}) and oxygen (C_{O2}) in the blended gas mix (17) which is used to determine the density of the gas mixture (ρₘᵢₓₜᵤᵣₑ) as follows:

ρₘᵢₓₜᵤᵣₑ=C_{He}ρ_{He}+C_{O2}ρ_{O2}

where ρ_{He} the density of helium gas, and ρ_{O2} the density of oxygen gas.

The pressure differential is ascertained using a venturi tube, flow nozzle, or orifice disposed within the flow path of the blended gas (17). This pressure differential together with the calculated density of the gas mixture (ρₘᵢₓₜᵤᵣₑ) is used to determine the overall flow rate of the blended gas mixture (17). Note that, adjusting the gas concentration of helium and oxygen will result in an adjustment in the flow rate of the blended gas (17) delivered to the patient.

FIG. 2 is a schematic diagram of another embodiment of a positive pressure support Heliox delivery system (100). Except for the inclusion of more advanced positive pressure support features and gas delivery adjustments, many of the other aspects and features of the alternate embodiment of the positive pressure support Heliox delivery system operate the same or substantially as described above in conjunction with the embodiment associated with FIG. 1. As such, the descriptions of the common elements and features will not be repeated here.

As seen in FIG. 2, the illustrated embodiment includes a negative pressure trigger valve (120), a sensor (125), and an exhaust valve (130). The sensor (125) is adapted to sense the pressure in the breathing circuit (140) and the controller (42) sends a command signal to the trigger valve (120) in response to the pressure in the breathing circuit (140) to effectuate operative control of the trigger valve (120). When the patient begins to inhale, a negative pressure condition is created in the breathing circuit (140). This negative pressure condition is detected by sensor (125). When the negative pressure condition exceeds a prescribed setpoint or threshold, the controller (42) delivers a command signal to open the trigger valve (120) and allow the blended gas (17) to flow to the patient. The negative pressure trigger setpoint or threshold is established as a user input to the system (100) and is generally selected to match the patient's inspiratory effort. The triggering or negative pressure condition is an adjustable parameter set by the user and ranges from about −0.1 cm H₂O to about −2.0 cm H₂O in increments of 0.1 cm H₂O depending on patient's age and condition.

Once triggered, the blended gas flow (17) with a variable positive pressure support of up to about 30 cm H₂O will be delivered through the trigger valve (120) to the breathing circuit (140) to help the patient breath more easily. The level of positive pressure support to the blended gas flow (17) is preferably a user defined parameter that is adjusted to attain the optimum therapeutic effect. Also, the speed or rate of inspiratory pressure increase to the designated level of positive pressure support is an adjustable parameter that is user defined parameter and preferably displayed on control unit display in tenths of a second. As discussed above, the level of positive pressure support generally ranges from about 0.0 cm H₂O to about 30.0 cm H₂O in increments of 1.0 cm H₂O.

The trigger valve (120) is operatively controlled by the control unit (42) to close at or near a breath termination setpoint or when the patient is likely no longer inhaling. As used herein, breath termination setpoint is an adjustable parameter that generally ranges from an inspiratory pressure at about 5% of peak inspiratory flow to an inspiratory pressure at about 75% of peak inspiratory flow and is preferably adjusted in increments of about 5%. Since the breath termination setpoint is tied to peak inspiratory flow, the closing of the trigger valve (120) occurs in response to the pressure in the breathing circuit (140) reaching the prescribed breath termination condition, as determined by sensor (125). For example, when the pressure in the breathing circuit (140) reaches the prescribed breathing termination setpoint or threshold, the trigger valve (120) closes or substantially closes such that there is little or no flow of the blended gas (17) to the patient until the negative pressure trigger setpoint or threshold is again detected.

Depending on the therapeutic need, the expiratory pressure may also be adjusted through the control unit (42) in the range of about 0.0 cm H₂O to about 10.0 cm H₂O. When the expiratory pressure in the breathing circuit (140) is greater than zero, a positive end-expiratory pressure (PEEP) condition is created. PEEP condition is commonly used in treatment of chronic obstructive pulmonary disease (COPD) and acute respiratory distress syndrome (ARDS) by physician to help patients improve oxygenation and increase lung volume.

In the preferred embodiment, the breathing circuit (140) includes a coaxial tube or two limb tube which separates the inspiratory and expiratory gas. When the patient inhales, the gas flows through one limb of the tube to the patient. When the patient exhales, the exhausted air flows through the other limb of the tube to the exhaust valve (130). The exhausted air is subsequently released or recycled. Additional sensors, such as an oximeter can be integrated into the breathing circuit to monitor patient oxygen saturation and use such data as inputs to the control unit (40) for controlling the flow rate and relative concentrations of oxygen and helium delivered to the patient. Using the patient oxygen saturation data, the user can adjust Heliox concentration accordingly to keep the patient's saturation around a prescribed level, most preferably about 90% or greater.

As discussed with reference to FIG. 1, the control unit (40) includes a microprocessor based controller (42), a control unit display (44), and a user interface (45). The microprocessor based controller (42) operatively controls both the gas blender (16) to adjust the blending of oxygen and Heliox and the control valve (18) to adjust the flow rate or pressure of the blended gas mixture (17) in response to user inputs as well as the measured parameters from the gas analyzer (16), flow meters (58) and associated sensors. The control unit display (44) is preferably an LCD type screen that displays gas delivery parameters such as flow rate of mixed gas, helium concentration of the blended gas, oxygen concentration of the blended gas, breathing curve of the patient, oxygen saturation of the patient, inspiratory positive pressure support, positive pressure adjustments, negative pressure triggers, alarm settings and indicators, and other system operating parameters. In addition to the user interface (45) described with reference to FIG. 1, the user inputs for the embodiment of FIG. 2 would further include setpoints related to the negative pressure trigger as well as other positive pressure support, such as breath termination setpoint or inspiration time, positive pressure increasing rate, inhalation volume control, etc. Through the user interface (45), the user inputs such parameters that are used to control or adjust the helium and oxygen concentrations, blended gas pressure and flow rate over time or according to patient's breathing pattern.

The control unit (42) is further adapted to allow the user to select a final gas composition and visually confirm it without the need to use external calibration charts. The user can also select a final blended gas flow rate independent of the blended gas flow composition. Preferably, the flow rate delivered to the patient ranges from little or no flow during expiratory phase up to a maximum of about 25 liters per minute during inspiratory phase. Finally, the use of the gas analyzer (16) allows the present system to distinguish the different Heliox concentrations and adjusts the blending accordingly to produce a blended flow (17) having the desired oxygen and helium concentrations as well as the desired output flow rate.

As should be appreciated from consideration of the above-described embodiments, the present Heliox delivery system and method with positive pressure support can be configured as a Continuous Positive Airway Pressure (CPAP) system or, more preferably, as a Bi-level Positive Airway Pressure (BiPAP) system. In the Continuous Positive Airway Pressure (CPAP) configuration, a continuous flow of gas is delivered into a patient's airway through a specially designed nasal mask or nasal prong. The continuous flow of air creates enough pressure when patient inhales to keep the patient's airway open. The Bi-level Positive Airway Pressure (BiPAP) configuration, on the other hand, varies the pressure during each breath cycle, as opposed to a CPAP type system which provides a continuous flow of gas under positive pressure. When the user inhales using a BiPAP configuration, the positive pressure support is sufficient to keep the patient's airway open. However, upon breath termination or when the user exhales using a BiPAP type configuration, the pressure of the incoming gas flow drops, making it much easier for the patient to breath. In the BiPAP configuration, the positive pressure support during the inspiratory phase is preferably between about 15 cm H₂O to about 30 cm H₂O whereas the preferred positive pressure support is much lower or non-existent during the expiratory phase, preferably between about 0 cm H₂O to about 10 cm H₂O.

There are two main techniques for providing the positive pressure support to the patient. One technique is proportional assist ventilation (PAV) wherein the positive pressure support provided to the incoming gas stream increases in direct proportion to patient's breathing effort. Using the PAV technique, the greater the patient's effort, the greater the pressure of breathing delivered by the machine. Another technique is proportional positive airway pressure (PPAP). In the PPAP technique, the pressure of incoming gas stream provided to the patient is a function of the patient's flow rate. Either of these techniques can be used in conjunction with the present Heliox delivery system and method. U.S. Pat. No. 6,532,956 describes a process or method and system that use one or more of the parameters involved in the PAV or PPAP pressure calculation to manage the flow of air to the patient. Similar such techniques would be useful in managing the Heliox and oxygen gas flow to the patient in conjunction with the presently disclosed system.

The Heliox delivery system as shown and described with reference to FIGS. 1 and 2 is adapted to mix Heliox gas and oxygen gas to therapeutically effective concentrations. For example, helium gas concentration is preferably in the range of about 0% to about 90% more preferably between about 50% and 79%. Conversely, the oxygen concentration is preferably maintained in the range of about 50% or more to about 10%. The helium and oxygen concentrations are adjusted by a preset algorithm programmed within the control unit. Such algorithms may, for example, vary the helium concentration over time or according to inspiration or expiration pattern of the patient. Since the system allow FiO2 level lower than 21%, the patient's oxygen saturation level should be monitored using an integrated or independent sensor or device. Preferably, it is desirable to maintain the patient's oxygen saturation at 90% or greater while keeping helium concentration in the gas mixture as high as possible. The Heliox delivery system can deliver the blended gas mixture at adjustable flow rates depending on the patient condition and needs.

The present system and method for Heliox delivery with positive pressure support is particularly useful for the treatment of chronic obstructive pulmonary disease (COPD) and other diseases involving airway resistance such as airway obstruction, asthma, postextubation stridor, cystic fibrosis, croup, respiratory failure, bronchiolitis, acute respiratory distress syndrome (ARDS), lung injury, etc.

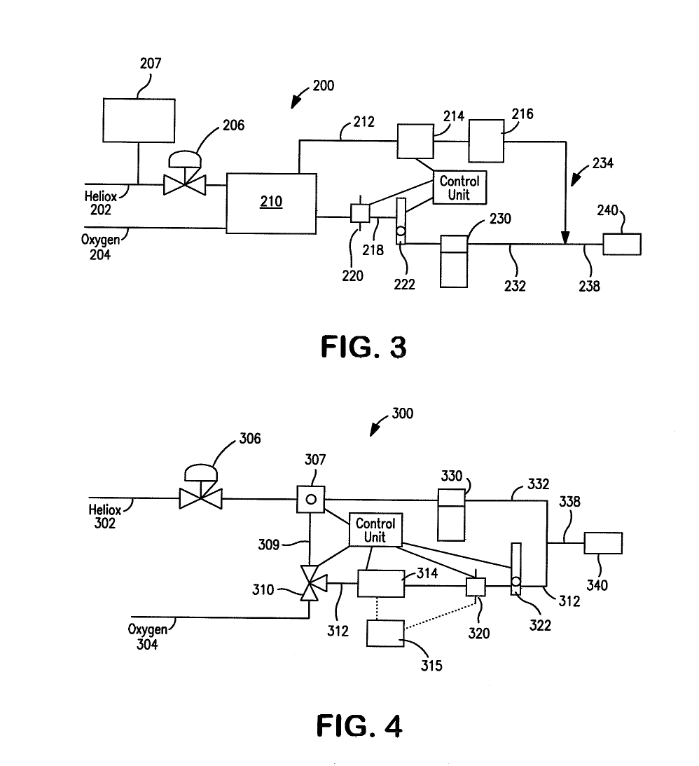

The present Heliox delivery system and method can also be useful for aerosolized drug delivery. Embodiments of the Heliox delivery system useful for aerosolized drug delivery are depicted in FIGS. 3 and 4. As depicted in FIG. 3, the Heliox delivery system (200) mixes Heliox (202) and oxygen (204) from appropriate gas sources. The Heliox (or helium) gas source is preferably a high-pressure gas source such as a cylinder or tank and having a regulator (206) and alarm (207) operatively coupled thereto. The system (200) depicted in FIG. 3 shows the oxygen gas (204) comes from a 50 psig line (e.g. wall outlet in a hospital), but it could also be equipped with a regulator similarly to the Heliox line.

The two gas lines connect to the inlets of the gas blender (210). The output of the gas blender (210) is separated in two lines. The first output line (212) of the blended gas goes to the gas analyzer (214). Since the first output line (212) does not have a flow meter disposed therein, it should be of a restricted calibrated diameter to limit the flow rate to a relatively small value (e.g. 5.0 liters per minute). The first output line (212) is thereafter heated using a heating filament (216) in the tubing or other heating means to produce a heated gas flow (234) at temperatures not to exceed about 60° C., and preferably to temperatures between about 25° C. and 35° C.

The second output line (218) from the gas blender (210) goes to a flow sensor (220) and a flow meter (222) responsive to measurements from the gas analyzer (214), as described above. The blended gas in the second output line (218) proceeds to a nebulizer (230) to produce a drug aerosol (232) that is subsequently mixed with the heated gas flow (234) of the first output line (212). The resulting blended gas output (238) with drug aerosol is administered to the patient via a breathing circuit (240) which preferably includes a facemask, nasal cannula, or any other existing delivery device (not shown).

An alternative Heliox delivery system (300) is depicted in FIG. 4. In this embodiment the Heliox gas (302) is routed through a regulator (306) and a two way selector (307) before any mixing with oxygen gas (304) in the gas blender (310). The selector (307) can be set to a first position or breathing position where the Heliox gas goes to the gas blender (310) to be mixed with oxygen gas (304) and eventually administered to the patient via breathing circuit (340). The desired concentration of the helium gas and oxygen gas as well as the flow rates are selected and provided as user inputs to a microprocessor based control unit (not shown). Alternatively, these could be manually inputted and controlled via knobs and dials on the blender (310) and the flowmeter (322) and confirmed visually on the outputs of the gas analyzer (314) and the flow rate indicator (315). Downstream of the gas blender (310), the line goes through a gas analyzer (314) and flow rate indicator (315). The flow in the line is subsequently metered and controlled using a flow meter (322) and a flow sensor (320) as described previously.

The selector (307) can also be set to a second position or drug delivery position where a portion of the Heliox gas is routed to a nebulizer (330) for the purpose of creating a drug aerosol (332) entrained in Heliox, preferably at a fixed flow rate. Preferably, the drugs should be aerolized and delivered with the highest concentrations of helium practical since, due to the lower gas density of Helium as compared to oxygen or air, the propensity for early drug deposition in the respiratory tract is reduced. The Heliox flow into and out of the nebulizer (330) is preferably fixed by a small calibrated inlet diameter of the nebulizer (330) so as to create sonic flow conditions. The remaining flow of Heliox (309) is mixed with the flow of oxygen (304) in the gas blender (310) to create a blended gas flow (312). The blended gas flow (312) exiting the gas blender (310) is subsequently combined with the aerosolized drug flow (332) to create an output flow (338) for administration to the patient via the breathing circuit (340).

Overall operation of the system (300) depicted in FIG. 4 is both flexible and simple, since the user only has to: (i) connect the system to the oxygen source and Heliox source; (ii) select the operation mode (e.g. drug delivery or breathing); (iii) select the relative gas concentrations; and (iv) select the desired flow rate and possibly the positive pressure support characteristics if needed. As described above, selecting of the relative gas concentrations, desired flow rates, and positive pressure support characteristics is preferably accomplished via a user interface associated with a control unit (not shown).

Heliox therapy is optimized with high helium content. Indeed, the lower density of the helium gas compared to air or nitrogen tends to reduce the work of breathing by most patients. Using Heliox also increases convective flows into the peripheral lung of the patient which promotes increased diffusional flows, thus leading to more effective gas exchange. Clinical studies suggest a consistent pattern of lower resistance and improved ventilation with Heliox, including larger tidal volumes and more complete exhalation, corresponding to improved pulmonary CO₂ removal.

Heliox has a similar ability to carry a medicinal aerosol as air or oxygen since the effect of gas density on aerodynamic force will be minimal for drug aerosols having particle sizes typical for inhalation drug delivery. The increased momentum associated with the Heliox flows will therefore effectively drive the aerolized drug particles deeper into the lung. Scintigraphy studies have confirmed that aerosol drug deposition in the peripheral lung increases proportionally with decreased resistance. Exercise studies have demonstrated that subjects breathe at higher rates and with higher tidal volumes when inhaling Heliox gas as opposed to air, which under ideal drug delivery conditions would allow for more drugs to be delivered to the lungs as well.

In light of the above teachings referenced in the Journal of Aerosol Medicine 2004 (volume 17, number 4, pp 299-309) by Corcoran and Gamard, it is often desirable to maximize the helium content of the Heliox gas to enhance the aerosolized drug delivery to the lungs on the one hand while on the other hand, it is also desirable to blend the Heliox mixture with pure oxygen to increase the patient oxygenation. In an effort to satisfy both objectives, it is proposed to optimize the drug deposition in the patient lung by using a nebulizer powered with a high helium content Heliox gas (i.e. aerosol phase) operated in alternating pulses or otherwise interposed with an aerosol-free Heliox gas flows possibly containing a higher oxygen content (i.e. aerosol-free phase).

In practice, the volumetric ratio of the aerosol phase to the aerosol-free phase can be from about 0.1 to about 10.0, with a preferred range of about 0.2 to about 1.0. The duration of the aerosol pulses can be from about 10% of the duration of a breathing cycle (inhalation plus exhalation) up to a duration of about 1000 breathing cycles, with a preferred range of 20% of the duration of a breathing cycle up to a duration of about 100 breathing cycles and still more preferably a duration of between about 1 to 30 breathing cycles. The oxygen concentration for the aerosol phase is preferably between about 0% to 50% with a preferred concentration of about 10% to 30%. Comparatively, the oxygen concentration in the aerosol-free phase of gas delivery is preferably between about 10% to 100% with a preferred concentration of about 20% to 50% with the balance being mainly helium.

The embodiment of FIG. 4 is useful to implement the alternating pulse or phased delivery scheme. The selector can be commanded or controlled to automatically switch between the aerosol phase where pure helium or a Heliox mix (e.g. 80% helium/20% oxygen or 90% helium/10% oxygen) is used to power a nebulizer directly and the aerosol-free phase, which blends oxygen gas and Heliox gas at a prescribed concentration levels. Operation of the selector can be controlled via a two-way solenoid valve controlled by a variable time delay relay or other suitable control mechanism.

While the present invention has been described with reference to a preferred embodiment, as will occur to those skilled in the art, numerous changes, additions and omissions may be made without departing from the spirit and scope of the present invention, as defined by the appended claims.

What is claimed is:

1. A positive pressure support Heliox delivery system comprising:
at least two inlet ports, one of which is coupled to a source of helium containing gas and another coupled to a source of oxygen containing gas; a gas blender in flow communication with the two inlet ports;
a flow valve disposed downstream of the gas blender;
a gas analyzer disposed downstream of the flow valve;
a flow meter disposed downstream of the flow valve;
a breathing circuit including at least one pressure sensor disposed downstream of the flow valve; and
a control unit adapted to receive inputs from the gas analyzer, the pressure sensor, the flow meter, and user inputs, the control unit further adapted to operatively control the gas blender to adjust the blending of oxygen gas and helium containing gas and to control the flow valve to adjust the blended gas flow rate and pressure in response to the inputs from the gas analyzer, pressure sensor, flow meter, and user inputs.

2. The positive pressure support Heliox delivery system of claim 1 wherein the helium containing gas is a Heliox gas blend.

3. The positive pressure support Heliox delivery system of claim 1 wherein the flow rate or pressure of the blended gas are adjusted in response to the pressure in the breathing circuit.

4. The positive pressure support Heliox delivery system of claim 1 wherein the flow rate or pressure of the blended gas is adjusted in response to a measured flow rate in the breathing circuit.

5. The positive pressure support Heliox delivery system of claim 1 wherein the flow valve is opened to deliver the blended gas to the breathing circuit when a negative pressure condition is created in the breathing circuit, as determined from the pressure sensor.

6. The positive pressure support Heliox delivery system of claim 1 wherein the flow valve is closed to interrupt delivery of the blended gas to the breathing circuit when a breath termination condition is created in the breathing circuit.

7. The positive pressure support Heliox delivery system of claim 1 wherein the control unit is adapted to automatically control the concentrations of helium and oxygen in the blended gas stream based on the breathing pattern of the patient or as a function of time.

8. The positive pressure support Heliox delivery system of claim 1 wherein the gas analyzer measures the concentration of oxygen gas and independently measures the concentration of the helium gas.

9. The positive pressure support Heliox delivery system of claim 8 wherein the concentration in the gas stream is automatically controlled via a gas mixing element based on the results provided by the gas analyzer.

10. A method for delivering Heliox to a patient via a breathing circuit, the method comprising the steps of:
blending a helium containing gas and oxygen gas to form a blended gas;
controlling the flow rate and pressure of the blended gas;
analyzing the concentrations of oxygen and helium in the blended gas;
ascertaining the pressure and flow rate in the breathing circuit;
operatively controlling the blending of the helium containing gas and oxygen gas in response to the readings from the oxygen and helium concentrations.

11. The method of claim 10 wherein the step of adjusting of the flow rate and pressure of the blended gas mixture delivered to a patient is in response to the ascertained pressure and flow rate in the breathing circuit.

12. The method of claim 10 wherein the helium containing gas is a Heliox gas blend.

13. The method of claim 10 wherein the blended gas flow to the breathing circuit is initiated when a negative pressure condition is detected in the breathing circuit.

14. The method of claim 10 wherein the blended gas flow to the breathing circuit is interrupted when a breath termination condition is detected in the breathing circuit.

15. The method of claim 10 wherein the step of analyzing the concentrations of oxygen and helium in the blended gas further comprises independently ascertaining the concentration of the helium gas and the concentration of the oxygen gas.

16. The method of claim 10 wherein the step of blending a helium containing gas and oxygen gas to form a blended gas further comprises blending a helium containing gas and oxygen gas to form a hypoxic blended gas having a helium concentration of 90% or greater.

17. The method of claim 10 wherein the step of operatively controlling the blending of the helium containing gas and oxygen gas further comprises cycling between a hypoxic blended gas having a helium concentration of 80% or greater and a blended gas having an oxygen concentration of 21% or greater.
